(19) United States
(12) Patent Application Publication
(10) Pub. No.: US 2012/0025088 A1
(43) Pub. Date: Feb. 2, 2012

(54) RADIOLOGICAL IMAGE RADIOGRAPHING AND DISPLAYING METHOD AND APPARATUS

(75) Inventor: Hiroki NAKAYAMA, Kanagawa (JP)

(73) Assignee: FUJIFILM CORPORATION, Tokyo (JP)

(21) Appl. No.: 13/190,064

(22) Filed: Jul. 25, 2011

(30) Foreign Application Priority Data
Jul. 28, 2010 (JP) 2010-168916

(51) Int. Cl. A61B 6/02

(52) U.S. Cl. 250/370.08; 378/41

(57) ABSTRACT

To display a stereoscopic image enabling a viewer to appropriately view the entire image stereoscopically even when the inclination of a detection plane of a radiological image detector is changed depending on the radiographing directions of radiological images constituting a stereoscopic image. The inclination of a detection plane of a radiological image detector is changed depending on radiographing directions and a stereoscopic image is displayed using projected radiological images obtained by projecting radiological images detected on the inclined detection plane onto a plane perpendicular to a desired viewpoint direction of the stereoscopic image.

### BACKGROUND OF THE INVENTION

1. Field of the Invention

The present invention relates to radiological image radiographing and displaying method and apparatus in which a stereoscopic image is displayed using radiological images, which are acquired by radiographing a subject from different radiographing direction, from the different radiographing directions.

2. Description of the Related Art

It has been known that an image can be viewed stereoscopically using parallax by combining and displaying plural images. Such an image (hereinafter, referred to as a stereoscopic image or a stereo image) that can be viewed stereoscopically is generated based on plural images with parallax acquired by taking the image of the same subject from different directions.

Moreover, such way of generating stereoscopic image is utilized not only in the field of digital cameras and televisions but also in the field of radiographing a stereoscopic image. That is, a test subject is irradiated with radiation from different directions, the radiation passing through the test subject is detected by a radiological image detector to acquire plural radiological images having parallax, and a stereoscopic image is generated based on the radiological images. By generating a stereoscopic image in this way, it is possible to observe a radiological image with a sense of depth and thus to observe a radiological image more suitable for diagnosis.

In general, in a radiological image radiographing apparatus, a grid used to remove scattered radiation is disposed on a detection plane side of a radiological image detector so as to prevent the radiation scattered by a subject from being incident on the radiological image detector.

The above-mentioned scattered radiation also causes a problem in a radiological image radiographing apparatus radiographing a stereoscopic image. For example, JP2004-73449A and JP2004-81330A propose a radiological image radiographing apparatus radiographing a stereoscopic image using a grid.

### SUMMARY OF THE INVENTION

However, in the radiological image radiographing apparatus radiographing a stereoscopic image, radiation is applied in the radiographing directions forming a predetermined convergence angle. Accordingly, for example, when the direction of the grid is fixed relative to the radiographing direction, the radiation transmitting direction of the slits in the grid may not be parallel to the optical axis direction of the radiation, thereby causing the deterioration in image quality of a radiological image.

Therefore, for example, it is considered that the direction of a radiation-receiving plane of the grid and the direction of a detection plane of the radiological image detector should be changed so as to make the radiation-receiving plane of the grid substantially perpendicular to the radiographing direction. In this case, as shown in FIG. 8, since a subject is located on and fixed in position to a radiography platform in spite of the change in direction of the detection plane of the radiological image detector, a radiological image P detected on the detection plane of the radiological image detector has different distances between the subject M and the detection plane on the right side and the left side in FIG. 8. The image on the right side is sparse and the image on the left side is dense, thereby generating a radiological image with convergence.

When such convergence is added to the radiological images forming a stereoscopic image, a shift between two radiological images will be greater than the predetermined parallax at the edge of the stereoscopic image, thereby making it difficult to view the image stereoscopically.

The present invention has been made in view of the above-mentioned problems and an object of the invention is to provide radiological image radiographing and displaying method and apparatus in which a stereoscopic image enabling a viewer to appropriately view the entire image stereoscopically can be displayed even when the inclination of a detection plane of a radiological image detector is changed depending on the radiographing directions of radiological images constituting the stereoscopic image.

According to an aspect of the present invention, there is provided a radiological image radiographing and displaying method of irradiating a subject with radiation from plural different radiographing directions, detecting a radiological image in each radiographing direction resulting from the irradiation with the radiation by the use of a radiological image detector, and displaying a stereoscopic image using the detected radiological images, wherein the inclination of a detection plane of the radiological image detector is changed depending on the radiographing directions, and the stereoscopic image is displayed using projected radiological images obtained by projecting the radiological images detected on the detection plane of the inclined radiological image detector onto a plane perpendicular to a desired viewpoint direction of the stereoscopic image, respectively.

According to another aspect of the present invention, there is provided a radiological image radiographing and displaying apparatus including: a radiation irradiating unit that irradiates a subject with radiation from plural different radiographing directions; a radiological image detector that detects a radiological image in each radiographing direction resulting from the irradiation with the radiation by the radiation irradiating unit; a display unit that displays a stereoscopic image using the plural radiological images detected by the radiological image detector; and a inclination changing mechanism that changes the inclination of a detection plane of the radiological image detector depending on the radiation directions, wherein the display unit displays the stereoscopic image using projected radiological images which is obtained by projecting the radiological images detected on the detection plane of the radiological image detector inclined by the inclination changing mechanism onto a plane perpendicular to a desired viewpoint direction of the stereoscopic image, respectively.

In the radiological image radiographing and displaying apparatus, a grid in which a part transmitting the radiation and a part absorbing the radiation are alternately arranged may be disposed on a detection plane side of the radiological image detector, and the inclination changing mechanism may change the inclination of a radiation-receiving plane of the grid along with the detection plane of the radiological image detector. Here, the detection plane side means a side on which a device having a function of generating charges or light in response to the irradiated radiation exists closer to the radiation irradiating unit side of the radiological image detector.

The radiological image radiographing and displaying apparatus may further includes an image compensating unit that generates the projected radiological images which are obtained by projecting the radiological images detected on the detection plane onto a plane perpendicular to a desired viewpoint direction of the stereoscopic image.

The image correcting unit may generate the projected radiological images based on the angles of the radiographing directions.

The plane perpendicular to the desired viewpoint direction of the stereoscopic image may be set to be closer to the radiation irradiating unit side than the detection plane of the radiological image detector.

The radiological image radiographing and displaying apparatus may further include a radiography platform on which the subject is located, and the direction of a subject-located plane of the radiography platform may be fixed.

The plane perpendicular to the desired viewpoint direction of the stereoscopic image may be set to the subject-located plane.

The radiological image detector and the grid may be contained in a single casing.

The casing may rotate depending on the radiographing directions.

In the radiological image radiographing and displaying method and apparatus according to the present invention, since the inclination of the detection plane of the radiological image detector is changed depending on the radiographing directions and the projected radiological images are generated by projecting the radiological images detected on the inclined detection plane onto the plane perpendicular to the desired viewpoint direction of the stereoscopic image, it is possible to display a stereoscopic image enabling a viewer to appropriately view the entire stereoscopic image by displaying the stereoscopic image using the projected radiological images without convergence.

### BRIEF DESCRIPTION OF THE DRAWINGS

FIG. 1 is a diagram schematically illustrating the configuration of a breast image radiographing and displaying system employing a stereoscopic image radiographing and displaying apparatus according to an embodiment of the present invention.

FIG. 2 is a diagram illustrating an arm section of the breast image radiographing and displaying system shown in FIG. 1 as viewed from the right side of FIG. 1.

FIG. 3 is a block diagram schematically illustrating the internal configuration of a computer of the breast image radiographing and displaying system shown in FIG. 1.

FIG. 4 is a flowchart illustrating the operation of the breast image radiographing and displaying system employing the stereoscopic image radiographing and displaying apparatus according to the embodiment of the present invention.

FIG. 5 is a diagram illustrating a method of projecting a radiological image detected on a detection plane of a radiological image detector onto a virtual projection plane.

FIG. 6 is a diagram illustrating another method of projecting a radiological image detected on the detection plane of the radiological image detector onto the virtual projection plane.

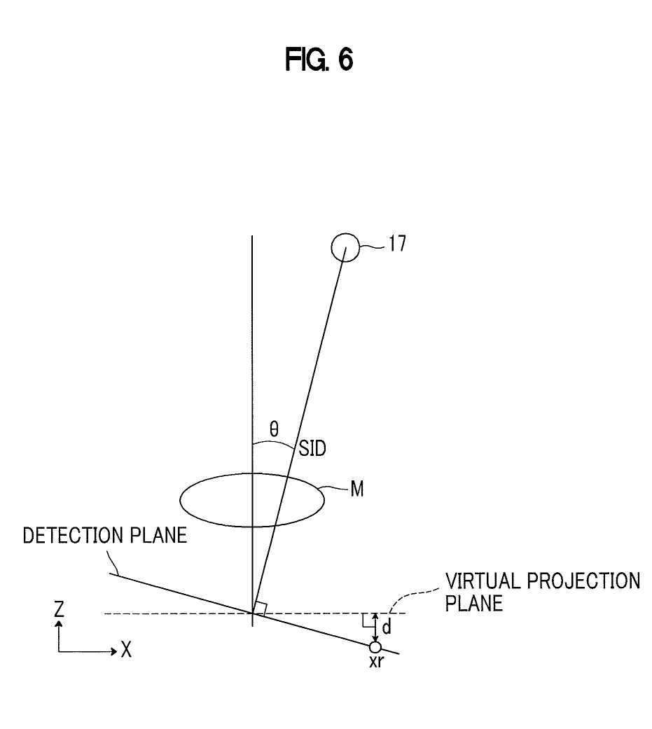

FIG. 7 is a diagram illustrating still another method of projecting a radiological image detected on the detection plane of the radiological image detector onto the virtual projection plane.

FIG. 8 is a diagram used to explain a problem when the inclination of the detection plane of the radiological image detector is changed depending on the radiographing direction of the radiological image.

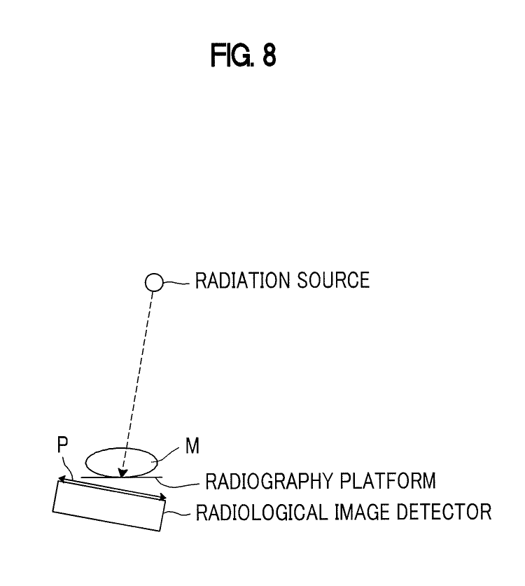

### DESCRIPTION OF THE PREFERRED EMBODIMENTS

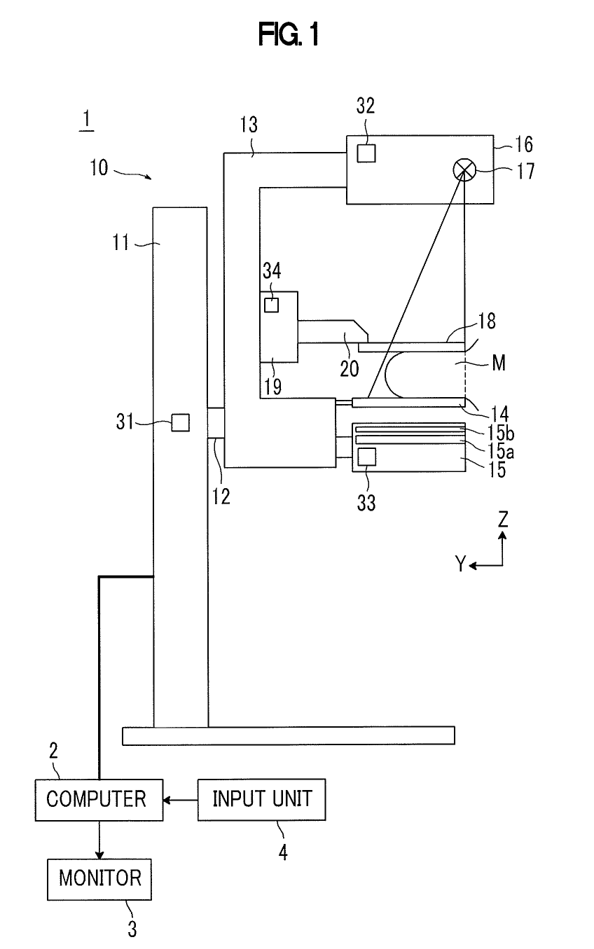

Hereinafter, a breast image radiographing and displaying system employing a radiological image radiographing and displaying apparatus according to an embodiment of the present invention will be described with reference to the accompanying drawings. FIG. 1 is a diagram schematically illustrating the entire configuration of the breast image radiographing and displaying system according to this embodiment.

As shown in FIG. 1, the breast image radiographing and displaying system 1 includes a breast image radiographing apparatus 10, a computer 2 connected to the breast image radiographing apparatus 10, and a monitor 3 and an input unit 4 connected to the computer 2.

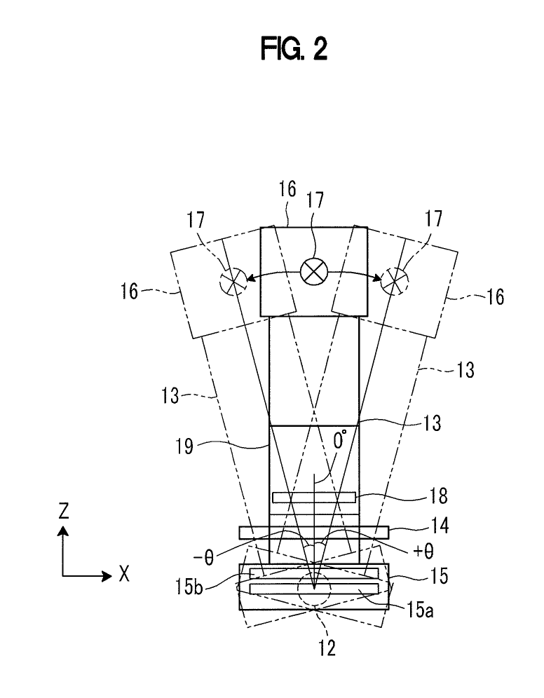

As shown in FIG. 1, the breast image radiographing apparatus 10 includes a base 11, a rotating shaft 12 that can move relative to the base 11 in the vertical direction (Z direction) and that can rotate, and an arm section 13 connected to the base 11 via the rotating shaft 12. FIG. 2 shows the arm section 13 as viewed from the right side in FIG. 1.

The arm section 13 is C-shaped, and has a radiography platform 14 and a radiographing unit 15 attached to one end and a radiation irradiating unit 16 attached to the other end so as to face the radiography platform 14. The rotation and the movement in the vertical direction of the arm section 13 are controlled by an arm controller 31 built in the base 11.

The radiography platform 14 is formed of a material transmitting radiation and is configured to be rotatable about the arm section 13. When the arm section 13 rotates relative to the base 11, the direction of the radiography platform 14 can be fixed relative to the base 11.

The radiographing unit 15 includes a radiological image detector 15a such as a flat panel detector, a grid 15b, and a detector controller 33 that controls the reading of a charge signal from the radiological image detector 15a. The radiography platform 14 includes a circuit board or the like in which a charge amplifier converting the charge signal read from the radiological image detector 15a into a voltage signal, a correlation double sampling circuit sampling the voltage signal output from the charge amplifier, an AD converter converting the voltage signal into a digital signal, and the like are disposed.

The radiographing unit 15 is configured to rotate about the arm section 13 and the inclination thereof can be changed depending on the radiographing directions of two radiological images constituting a stereo image. The detector controller 33 of the radiographing unit 15 acquires the radiographing directions of two radiological images and rotates and inclines the radiographing unit 15 so that the detection plane of the radiological image detector 15a and the radiation-receiving plane of the grid 15b are substantially perpendicular to the radiographing directions at the time of radiographing the radiological images.

The radiological image detector 15a can repeatedly write and read the radiological images and may employ a so-called direct-type radiological image detector being directly subjected to the irradiation with radiation and generating charges or may employ a so-called indirect-type radiological image detector converting radiation into a visible ray and converting the visible ray into a charge signal. As a method of reading a radiological image signal, a so-called TFT reading method of reading a radiological image signal by turning on or off a TFT (Thin Film Transistor) switch or a so-called optical reading method of reading a radiological image signal by applying a reading ray can be preferably employed. However, the present invention is not limited to these methods but other methods may be employed.

The grid 15b serves to remove scattered radiation and has a configuration in which a member absorbing radiation and a member transmitting radiation are alternately arranged to form a stripe shape or a grid shape.

A radiation source 17 and a radiation source controller 32 are received in the radiation irradiating unit 16. The radiation source controller 32 can control the time to emit radiation from the radiation source 17 and radiation generating conditions (such as the tube current, the time, and the product of tube current and time) in the radiation source 17.

The central portion of the arm section 13 is provided with a compression plate 18 being disposed above the radiography platform 14 and pressing a breast, a support 20 supporting the compression plate 18, and a moving mechanism 19 moving the support 20 in the vertical direction (Z direction). The position and the pressing pressure of the compression plate 18 are controlled by a compression plate controller 34.

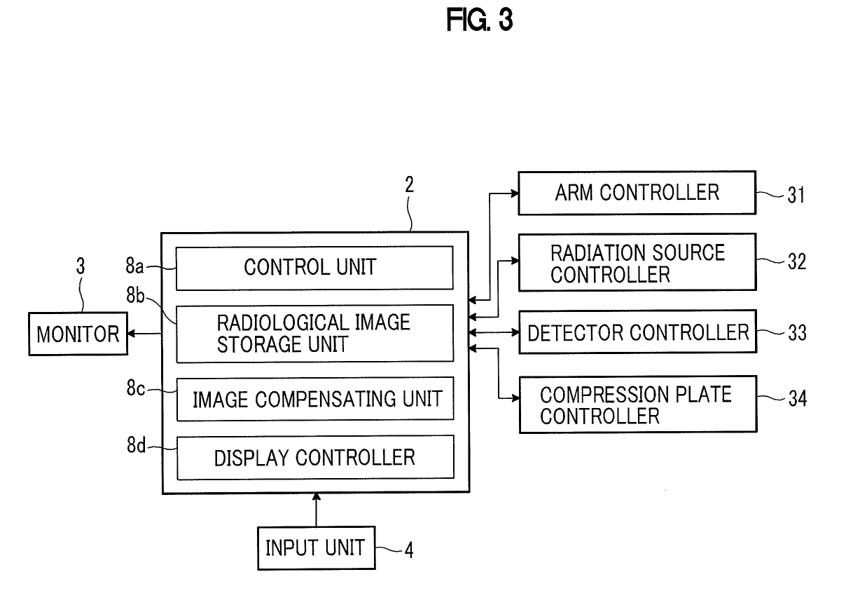

The computer 2 includes a central processing unit (CPU) and a storage device such as a semiconductor memory, a hard disk, or an SSD. A control unit 8a, a radiological image storage unit 8b, an image compensating unit 8c, and a display controller 8d shown in FIG. 3 are constructed by these hardware components.

The control unit 8a outputs a predetermined control signal to various controllers 31 to 35 and controls the entire system. The specific control method will be described later.

The radiological image storage unit 8b stores a radiological image signal acquired by the radiological image detector 15a in advance.

The image compensating unit 8c corrects the radiological images detected on the detection plane of the radiological image detector 15a inclined depending on the radiographing directions and generates projected radiological images which are obtained by projecting the radiological images onto a plane perpendicular to a desired viewpoint direction of a stereoscopic image. In this embodiment, it is assumed that the viewpoint direction is set to the direction of 0° and the image compensating unit 8c generates the projected radiological images by projecting the radiological images onto the plane perpendicular to the direction of 0°, that is, onto the breast-located plane of the radiography platform 14. The method of generating the projected radiological images will be described later in detail. In this embodiment, it is described that the viewpoint direction is set to the direction of 0°, but may be set to other directions.

The display controller 8d displays a stereoscopic image of a breast on the monitor 3 based on two projected radiological image signals generated by the image compensating unit 8c.

The input unit 4 includes pointing devices such as a keyboard or a mouse and receives the input of radiographing conditions including the convergence angle, the input of a radiographing start instruction, or the like from a radiographer.

The monitor 3 is configured to display a stereo image using two radiological image signals output from the computer 2 at the time of radiographing a stereo image. As the configuration displaying a stereo image, a configuration displaying a stereo image by displaying the radiological images based on two radiological image signals, for example, using two screens, allowing one radiological image incident on an observer's right eye using a half mirror or a polarizing glass and allowing the other radiological image incident on the observer's left eye can be employed. Alternatively, a stereo image may be generated by dislocating, superimposing and displaying two radiological images by a predetermined magnitude of parallax and observing the resultant image with a polarizing glass may be employed, or a stereo image may be generated by displaying two radiological images by the use of a 3D liquid crystal display enabling a stereoscopic view like a parallax barrier method or a lenticular method.

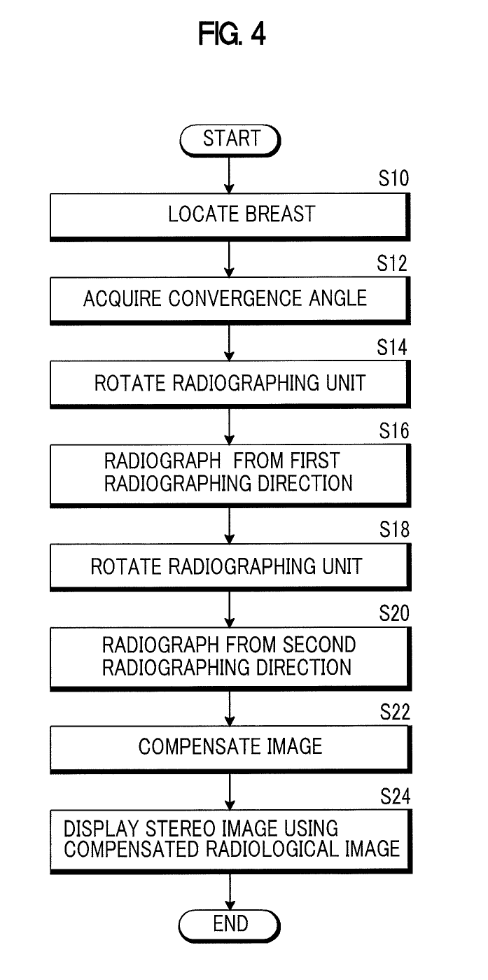

The operation of the breast image radiographing and displaying system according to this embodiment will be described with reference to the flowchart shown in FIG. 4.

First, a patient's breast M is located on the radiography platform 14 and the breast M is pressed with a predetermined pressure by the compression plate 18 (S10).

A radiographer inputs various radiographing conditions and then inputs a radiographing start instruction through the use of the input unit 4.

When the radiographing start instruction is input through the use of the input unit 4, first, the control unit 8a reads a preset convergence angle θ for radiographing a stereoscopic image and outputs the information of the read convergence angle θ to the arm controller 31 and the detector controller 33 (S12). In this embodiment, it is assumed that θ=±2° is stored in advance as the information of the convergence angle θ, but the present invention is not limited to this configuration and any convergence angle can be set by the use of the input unit 4.

When the arm controller 31 receives the information of the convergence angle θ output from the control unit 8a, the arm controller 31 outputs a control signal based on the information of the convergence angle θ so that the arm section 13 rotates by +θ° about the direction perpendicular to the radiography platform 14, as shown in FIG. 2. That is, in this embodiment, the control signal is output so that the arm section 13 rotates by +2° about the direction perpendicular to the radiography platform 14.

On the other hand, when the detector controller 33 receives the information of the convergence angle θ output from the control unit 8a, the detector controller 33 causes the radiographing unit 15 to rotate based on the information of the convergence angle θ so that the detection plane of the radiological image detector 15a and the radiation-receiving plane of the grid 15b are substantially perpendicular to the radiographing direction of +θ°, as shown in FIG. 2 (S14).

In the above-mentioned states of the arm section 13 and the radiographing unit 15, one radiological image out of two radiological images constituting a stereo image is captured (S16). Specifically, the control unit 8a outputs a control signal to the radiation source controller 32 and the detector controller 33 so as to emit radiation and to read a radiological image signal. In response to this control signal, the radiation is emitted from the radiation source 17, the radiological image obtained by radiographing the breast in the direction of +2° is detected by the radiological image detector 15a, the radiological image signal is read by the detector controller 33, the read radiological image signal is subjected to a predetermined signal process, and the resultant signal is stored in the radiological image storage unit 8b of the computer 2.

Then, the arm controller 31 temporarily returns the arm section to the initial position, as shown in FIG. 2, and then outputs a control signal so as to rotate by −θ° about the direction perpendicular to the radiography platform 14 (S14). That is, in this embodiment, the control signal is output so that the arm section 13 rotates by −2° about the direction perpendicular to the radiography platform 14.

On the other hand, the detector controller 33 causes the radiographing unit 15 to rotate based on the information of the convergence angle θ so that the detection plane of the radiological image detector 15a and the radiation-receiving plane of the grid 15b are substantially perpendicular to the radiographing direction of −θ°, as shown in FIG. 2 (S 18).

In the above-mentioned states of the arm section 13 and the radiographing unit 15, the second radiological image is captured (S20).

Specifically, as described above, the control unit 8a outputs a control signal to the radiation source controller 32 and the detector controller 33 so as to emit radiation and to read a radiological image. In response to this control signal, the radiation is emitted from the radiation source 17, the radiological image obtained by radiographing the breast in the direction of −2° is detected by the radiological image detector 15a, the radiological image signal is read by the detector controller 33, the read radiological image signal is subjected to a predetermined signal process, and the resultant signal is stored in the radiological image storage unit 8b of the computer 2.

Two radiological image signals stored in the radiological image storage unit 8b are read by the image compensating unit 8c and projected radiological image signals are generated based on the radiological image signals (S22).

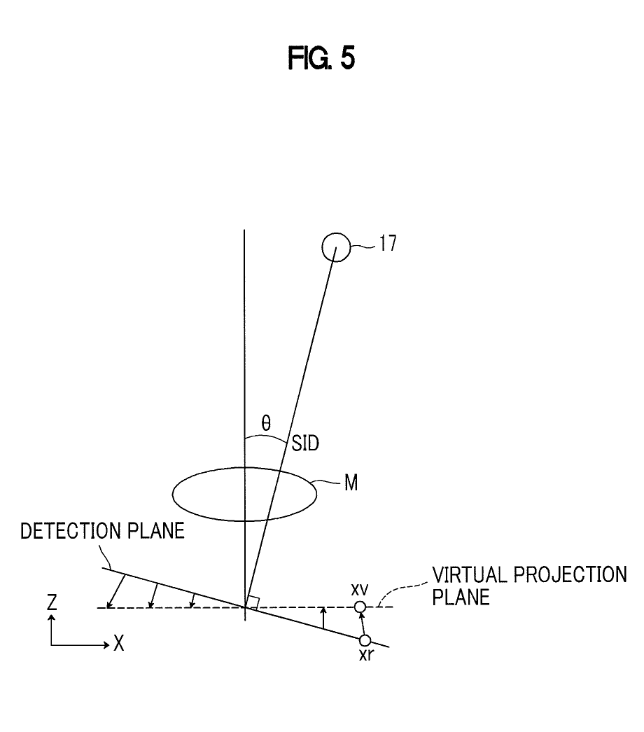

Specifically, as shown in FIG. 5, when the detection plane of the radiological image detector 15a is inclined by θ about the X direction, that is, when the radiographing angle is θ, the image compensating unit 8c calculates the x coordinate of a point xv by the use of the following expression so that a point xr of the radiological image detected on the detection plane of the radiological image detector is projected to the point xv on a virtual projection plane. The virtual projection plane shown in FIG. 5 is a plane perpendicular to a desired viewpoint direction of a stereo image and is the breast-located plane of the radiography platform 14 in this embodiment, as described above.

$$\begin{matrix}xv = S I D / \{ tan θ + ( S I D / xr ) \} \\ = ( S I D \times xr ) / ( xr \times tan θ + S I D )\end{matrix}$$

Here, xr represents the value of the x coordinate on the detection plane and SID represents the distance between the focal position of the radiation source 17 and the detection plane of the radiological image detector 15a.

The y coordinate of the point xv is calculated by the use of the following expression.

yv=(SID×yr)/(SID+d)

Here, d represents the length of a vertical line connecting the point xr to the virtual projection plane and is expressed by d=xr×sin θ. In addition, yr represents the y coordinate on the detection plane.

As described above, the image compensating unit 8c generates the projected radiological image signals obtained by projecting the radiological image signals onto the virtual projection plane by calculating the x coordinate and the y coordinate of the point xv, and outputs the projected radiological image signals to the display controller 8d.

The display controller 8d displays a stereo image of the breast on the monitor 3 based on the two input projected radiological image signals (S24).

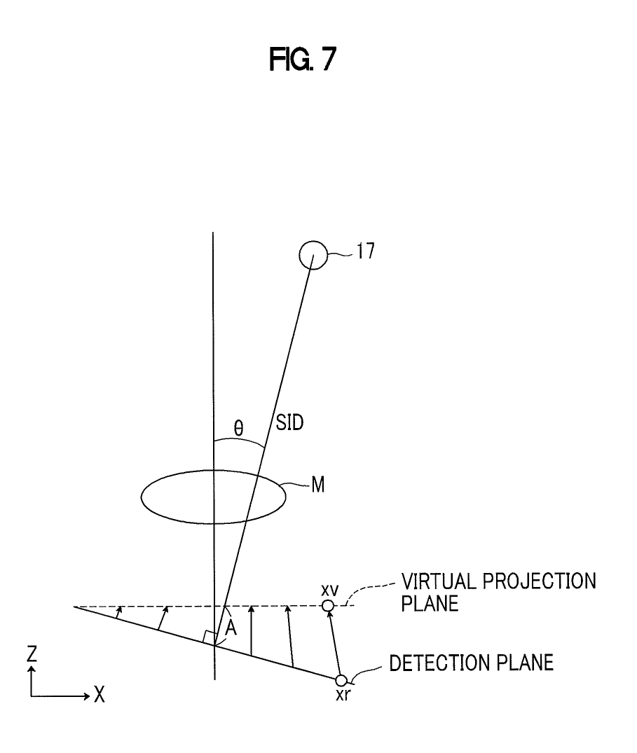

In this embodiment, when the image compensating unit 8c generates the projected radiological image signals, the virtual projection plane is set so that the central position of the detection plane of the radiological image detector 15a intersects the central position of the virtual projection plane, as shown in FIG. 5. However, the present invention is not limited to this configuration and it is preferable that the virtual projection plane is set to be closer to the radiation source 17 than the detection plane of the radiological image detector 15a, for example, as shown in FIG. 7. By setting the virtual projection plane in this way, since the projection from the point xr to the point xv can be carried out by a reduction process, the projected point can be made not to spread, thereby displaying an image which can be easily viewed stereoscopically. When the virtual projection plane is set in this way, the x coordinate of the point xv can be calculated by the use of the following expression.

$$\begin{matrix}xv = S I {D}^{′} / \{ tan θ + ( S I D / xr ) \} \\ = ( S I {D}^{′} \times xr ) / ( xr \times tan θ + S I D )\end{matrix}$$

Here, SID′ represents SID-A and A represents the length of the vertical line drawn from the central position of the detection plane to the virtual projection plane, as shown in FIG. 7.

The y coordinate of the point xv is calculated by the use of the following expression.

yv=(SID′×yr)/(SID′+d)

d=xr×sin θ

In the above description, the stereoscopic image displaying apparatus according to an embodiment of the present invention is applied to the breast image radiographing and displaying system. However, the subject in the present invention is not limited to the breast and the present invention can be applied to a radiological image radiographing and displaying system radiographing, for example, a chest region or a head region.

What is claimed is:

1. A radiological image radiographing and displaying method of irradiating a subject with radiation from plural different radiographing directions, detecting a radiological image in each radiographing direction resulting from the irradiation with the radiation by the use of a radiological image detector, and displaying a stereoscopic image using the detected radiological images,
wherein the inclination of a detection plane of the radiological image detector is changed depending on the radiographing directions, and
the stereoscopic image is displayed using projected radiological images obtained by projecting the radiological images detected on the detection plane of the inclined radiological image detector onto a plane perpendicular to a desired viewpoint direction of the stereoscopic image, respectively.

2. A radiological image radiographing and displaying apparatus comprising:
a radiation irradiating unit that irradiates a subject with radiation from plural different radiographing directions;
a radiological image detector that detects a radiological image in each radiographing direction resulting from the irradiation with the radiation by the radiation irradiating unit;
a display unit that displays a stereoscopic image using the plural radiological images detected by the radiological image detector;
a inclination changing mechanism that changes the inclination of a detection plane of the radiological image detector depending on the radiographing directions,
wherein the display unit displays the stereoscopic image using projected radiological images which is obtained by projecting the radiological images detected on the detection plane of the radiological image detector inclined by the inclination changing mechanism onto a plane perpendicular to a desired viewpoint direction of the stereoscopic image, respectively.

3. The radiological image radiographing and displaying apparatus according to claim 2, wherein a grid in which a part transmitting the radiation and a part absorbing the radiation are alternately arranged is disposed on the detection plane side of the radiological image detector, and
the inclination changing mechanism changes the inclination of a radiation-receiving plane of the grid and the detection plane of the radiological image detector.

4. The radiological image radiographing and displaying apparatus according to claim 2, further comprising an image compensating unit that generates the projected radiological images which are obtained by projecting the radiological images detected on the detection plane onto a plane perpendicular to a desired viewpoint direction of the stereoscopic image.

5. The radiological image radiographing and displaying apparatus according to claim 3, further comprising an image compensating unit that generates the projected radiological images which are obtained by projecting the radiological images detected on the detection plane onto a plane perpendicular to a desired viewpoint direction of the stereoscopic image.

6. The radiological image radiographing and displaying apparatus according to claim 4, wherein the image compensating unit generates the projected radiological images based on the angles of the radiographing directions.

7. The radiological image radiographing and displaying apparatus according to claim 5, wherein the image compensating unit generates the projected radiological images based on the angles of the radiographing directions.

8. The radiological image radiographing and displaying apparatus according to claim 2, wherein the plane perpendicular to the desired viewpoint direction of the stereoscopic image is set to be closer to the radiation irradiating unitside than the detection plane of the radiological image detector.

9. The radiological image radiographing and displaying apparatus according to claim 3, wherein the plane perpendicular to the desired viewpoint direction of the stereoscopic image is set to be closer to the radiation irradiating unit side than the detection plane of the radiological image detector.

10. The radiological image radiographing and displaying apparatus according to claim 4, wherein the plane perpendicular to a desired viewpoint direction of the stereoscopic image is set to be closer to the radiation irradiating unit side than the detection plane of the radiological image detector.

11. The radiological image radiographing and displaying apparatus according to claim 5, wherein the plane perpendicular to a desired viewpoint direction of the stereoscopic image is set to be closer to the radiation irradiating unit side than the detection plane of the radiological image detector.

12. The radiological image radiographing and displaying apparatus according to claim 2, further comprising a radiography platform on which the subject is located,
wherein the direction of a subject-located plane of the radiography platform is fixed.

13. The radiological image radiographing and displaying apparatus according to claim 3, further comprising a radiography platform on which the subject is located,
wherein the direction of a subject-located plane of the radiography platform is fixed.

14. The radiological image radiographing and displaying apparatus according to claim 4, further comprising a radiography platform on which the subject is located,
wherein the direction of a subject-located plane of the radiography platform is fixed.

15. The radiological image radiographing and displaying apparatus according to claim 5, further comprising a radiography platform on which the subject is located,
wherein the direction of a subject-located plane of the radiography platform is fixed.

16. The radiological image radiographing and displaying apparatus according to claim 8, further comprising a radiography platform on which the subject is located,
wherein the direction of a subject-located plane of the radiography platform is fixed.

17. The radiological image radiographing and displaying apparatus according to claim 12, wherein the plane perpendicular to the desired viewpoint direction of the stereoscopic image is set to the subject-located plane.

18. The radiological image radiographing and displaying apparatus according to claim 13, wherein the plane perpendicular to the desired viewpoint direction of the stereoscopic image is set to the subject-located plane.

19. The radiological image radiographing and displaying apparatus according to claim 3, wherein the radiological image detector and the grid are contained in a single casing.

20. The radiological image radiographing and displaying apparatus according to claim 19, wherein the casing rotates depending on the radiographing directions.
